(19) United States
(12) Patent Application Publication
(10) Pub. No.: US 2026/0076917 A1
(43) Pub. Date: Mar. 19, 2026

(54) COMPOSITION AND PROCESS FOR A LIPID NANO-PARTICLE DELIVERY OF OXYGEN AND VITAL METABOLIC COMPOUNDS FOR HEMORRHAGIC SHOCK

(71) Applicants: KaloCyte, Inc., Baltimore, MD (US); UNIVERSITY OF MARYLAND, BALTIMORE, Baltimore, MD (US)

(72) Inventors: William D. McGhee, Fenton, MO (US); Stephen C. Rogers, Ellicott City, MD (US); Allan Doctor, Towson, MD (US); Dipanjan Pan, State College, PA (US)

(21) Appl. No.: 19/303,206

(22) Filed: Aug. 18, 2025

Related U.S. Application Data
(60) Provisional application No. 63/684,007, filed on Aug. 16, 2024.

(51) Int. Cl. A61K 9/51; A61K 9/00; A61K 9/19; A61K 31/708; A61K 31/7084; A61K 38/42; A61K 47/40; A61K 47/42; A61P 7/00

(52) U.S. Cl. CPC A61K 9/5123; A61K 9/0019; A61K 9/19; A61K 31/708; A61K 31/7084; A61K 38/42; A61K 47/40; A61K 47/42; A61P 7/00

(57) ABSTRACT

Generation of lipid nano-particles containing purified human hemoglobin as an oxygen carrier was accomplished with the co-encapsulation of metabolic agents. Additionally, the nano-particles described are of the long-circulating variety (Pegylation of the exterior surface) which are more tolerable in vivo when compared against their non-pegylated counterparts. The generated nano-particles are lyophilizable, given proper formulation with lyo-protectants, which are storage stable solids ready for reconstitution prior to use.

### FIELD OF THE INVENTION

This invention relates to a composition and process for the generation of, and for the delivery of oxygen and metabolic factors/co-factors to organs in need due to insufficient ability of red blood cells to perform these functions.

### BACKGROUND OF THE INVENTION

Physiologically, the prevalent allosteric effector for hemoglobin in humans is 2,3-DPG (2,3-Diphosphoglycerate) while other phosphate-containing molecules have been identified as important effectors in other species. Other phosphate containing allosteric effectors include: IHP (inositol hexa-phosphate), sphingosine monophosphate, ATP (adenine triphosphate), and GTP (guanosine triphosphate), the latter two of which are believed to play important roles in fish and NAD (H)/NADP (H). In human RBC (red blood cells), 2,3-DPG and its variability dominate the allostery for Hb. Although ATP has a presence in RBC, its concentration (1 uM) and the chelation of ATP with Mg²⁺ ions (Mg2+ at 3 uM in RBC reduces the ability of ATP to bind to the Hb) significantly renders ATP to a lesser role as an allosteric effector. GTP is found in RBC but at low concentrations relative to ATP.

ATP and GTP are key physiological molecules for energy in many cellular functions. In hemorrhagic shock, a substantial loss of ATP from major organs occurs. Intravenous administration of ATP-MgCl₂ has ample president in partial restoration in organs such as the liver and kidney. The short circulatory stability of ATP hinders the effectiveness of direct systemic infusion. Liposomal entrapped ATP-MgCl₂ has been described to deliver the ATP to various organs during ischemia, including the liver during hemorrhagic shock.

### SUMMARY OF THE INVENTION

Generation of lipid nano-particles containing purified human hemoglobin as an oxygen carrier was accomplished with the co-encapsulation of metabolic agents. The purpose of the metabolic agents is two-fold: to provide for metabolic energy during metabolism in cells of injured organs, which were the consequence of loss of RBCs, and to act as an allosteric effector for the hemoglobin in its ability to transport oxygen. The allosteric effector provides for a change in the ability of the hemoglobin to bind hemoglobin, with a lower affinity in effected organs (higher p50, release of oxygen) while distinctly higher in the lung capillaries (lower p50, higher oxygen binding). Although available in RBCs, metabolic agents described in this application are not effective as allosteric effectors due to low relative concentrations. Blood substitutes of purified hemoglobin, or polymerized hemoglobin have been reported and contain allosteric effectors such as 2,3-DPG, IHP or pyroxidal-5-phosphate while failing to use the nucleoside metabolites, ATP, UTP, CTP, GTP, NADP (H), FADP etc. In this study we show that the use of nucleoside metabolic agents gives p50 values for hemoglobin, encapsulated in nano-particles, which are ideal for oxygen transfer. These nucleoside metabolic agents are also available for restoration of cellular functions in damaged cells of effected organs.

Additionally, the nano-particles described herein are of the long-circulating variety (Pegylation of the exterior surface) which are more tolerable in vivo when compared against their non-pegylated counterparts. The generated nano-particles are lyophilizable, given proper formulation with lyo-protectants, which are storage stable solids ready for reconstitution prior to use.

It is specifically noted that every combination and sub-combination of the features and embodiments described herein is considered to be part of the invention.

### DETAILED DESCRIPTION OF THE INVENTION

### Example 1: Formation of Lyophilized Lipids

DPPC DPPGNa, cholesterol, KC1003-acetate and DSPE-PEG (2000) are removed from freezer (−20 storage) and allowed to warm to RT. The following amounts were weighed (Table 1) into a 500 mL RB flask.

TABLE 1
Lipids for solid mixture

| Lipid | FW | Grams |
|---|---|---|
| DPPC | 734 | 6.09 |
| DPPGNa | 745 | 0.854 |
| Cholesterol | 386.6 | 1.73 |
| KC-1003 | 712 | 0.796 |
| DSPE-PEG(2000) | 2805 | 0.126 |

The lipids were then dissolved in t-BuOH (50 mL). The hazy solution of lipids in t-BuOH was transferred to a lyophilization jar and frozen in freezer at −80° C.

A lyophilizer chiller was set at <−95° C. and vacuum <150 millitorr. The jar with frozen lipids in t-BuOH was attached to lyophilizer and opened to full vacuum. The temperature and pressure in the lyophilizer were checked until steady state achieved 4-6 days. Lyo-cake stored at −80° C. until use in the hydration step.

### Example 2: Hydration of Lyo-Cake with Hb

Purified Hemoglobin thawed ahead of time and kept at 5° C. prior to use. (5 mM Hb in 10 mM phosphate buffered water at pH 6.5-6.75. The following additives to the Hb were weighed out: 1) ATP (adenosine triphosphate disodium salt) at 6.0 mg/mL of Hemoglobin and dissolved in WFI (at 500 mg/mL) 2) MgCl2 (anhydrous) at 0.36 mg/mL of Hemoglobin and dissolved in WFI (at 100 mg/mL)-exothermic dissolution 3) NaCl at 2.5 mg/mL of Hemoglobin 4) Sucrose at 10 mg/mL Hemoglobin and 5) HPCD (hydroxypropyl cyclodextrin) at 25 mg/mL Hemoglobin.

The Hemoglobin was filtered first through a 0.22 micron sterile filter followed by a 0.1 micron sterile filter in the biohood. To the filtered Hemoglobin add (in order) was added 1) ATP with swirling, 2) MgCl2 with swirling 3) NaCl as a solid, swirl to dissolve 4) Sucrose as a solid, swirl to dissolve 5) HPCD. The mixture was sonicated in a batch sonicator for 20 minutes at ambient temperature. Filter the Hb mixture through a 0.1 micron sterile filter and then a 0.03 sterile membrane filter.

200 mL of the filtered Hb mixture was added to the lyophilized lipids (lyophilized lipids produced as a powder, clumps broken before addition of Hb) at ambient temperature and at a rate that prevents formation of clumps. The mixture was stirred 100 rpm for a minimum 2 hrs at room temperature.

### Example 3: Extrusion of Hydrated Lipid Vesicles

The Hb/hydrated lipid vesicles from the hydration step was added to a 800 mL extruder at room temperature and passed through double stacked 0.8 micron membranes stacked membranes a2 times. The resulting mixture was then passed through, 2 times each, in sequence double stacked 0.6 micon, 0.4 micron, and finally 0.2 micron membranes. The resulting particle suspension was saturated with CO and chilled at 5° C.

### Example 4: Cross Flow for EM Purification

PBS, sterile filtered at pH 7.0 was used to remove unencapsulated materials using a 750 K NMWC fiber. Crude particles from extruder was pre-cooled to 4° C. in refrigerator, diluted with the PBS buffer and diafiltered with 7× volume turnover for full removal. The resulting material was concentrated to 100 mL using ultrafiltration. Concentrated retentate removed from reservoir and saturated with CO prior to storage at 4° C.

### Example 5: PEG-Post Insertion-KC240808-PP

To a 250 ml bottle was weighed 4.4 g DSPE-PEG (2000) and dissolved into 110 mL of purified water. The solution was filtered through a 0.1 micron sterile filter.

175 mL of the purified particle suspension of Hb (KC240808) and 100 mL of the 40 mg/mL solution of DSPE-PEG (2000) were added to a 1 L three neck flask fitted with a gas inlet, gas outlet (attached to a ballon) and a thermometer. With magnetic stirring the flask was filled with CO at ambient temperature. CAUTION: CO is highly poisonous and should only be used in a hood designated for use of CO and has an audible CO alarm in proximity to the hood

The CO was released into the fume hood and the fill-release steps repeated 3×. After the flask was purged, the ballon is partially inflated with CO. The mixture was heated in a water bath to 60° C. under the CO atmosphere for 1 hr. Reaction was allowed to cool to ice temperature (ice bath) under CO, once chilled the CO was removed and the resulting mixture purified by cross-low filtration to remove any residual DSPE-PEG (2000) using PBS.

TABLE 2
KC24808-PP Properties

| | Clean | | size | Zeta | | Total | | | % |
|---|---|---|---|---|---|---|---|---|---|
| | Encapsulated | EE | Malvern | Potential | | lipids | | PEG | Met |
| EM ID# | [Hb] uM | % | (nm) | ZV (mV) | PDI | mg/ml | Hb:lipid | % | Hb |
| KC240808-PP | 968 ± 18 | 99.3% | 211 | −32 | 0.13 | 64 | 0.98 | 2.3% | 0.4% |

### Example 6: Lyophilization of Cleam EM

2.74 g of HPCD (EMPROVE EXPERT Cyclodextrin HPB) and 1.82 g of BSA (Bovine Serum Albumin, low-endotoxin) were weighed out in a 250 ml bottle. This mixture was dissolved into 75 mL purified water for injection and then filtered through a 0.1 micron filter.

To a 20 mL sterile de-pyrogenated vial was added 5.3 mL of EM and 5.3 mL of the HPCD/BSA solution. The vials were argon purged prior to lyophilization. The lyophilization cycle (Table 3) was carried out giving Red solid lyophilized cakes which were stored at 5° C.

TABLE 3
Lyophilization Cycle

| Step Time | | | | | |
|---|---|---|---|---|---|
| (min) | Temperature | | Pressure | | Cycle |
| 0 | Ambient | | Atmospheric | | Pre-lyophilization |
| 30 | RT to −45° C. | | 500 | torr | Slow freezing |
| 240 | −45° | C. | 500 | torr | Freeze |
| 30 | −45° C. to | | 250 | mtorr | Ramp to first lyophilization |
| | −15° C. | | | | |
| 4000 | −15° | C. | 250 | mTorr | Cold lyophilization |
| 1000 | −15° | C. | 250 | mTorr | Second lyophilization |
| 1000 | 0° | C. | 250 | mTorr | Final “drying” |
| 15 | 5° | C. | 250 | mTorr | |
| End | 15° | C. | 600 | Torr | End of Lyophilization/ |

Backfill N2

### Example 7: ATP as Allosteric Effector and Metabolism Additive

5 mL of purified hemoglobin at 5 mM in 10 mM phosphate buffer at pH 6.75 is filtered through a 0.1 micron filter. To the filtered Hb is added as appropriate (see Table) HPCD, Glucose, NaCl, MgCl2 and ATP. The mixture is sonicated for 1 minute in a bath sonicator and then filtered through a 0.05 micron syringe filter.

Into a 20 mL reaction vessel is weighed 240 mg of solid lipid mixture (DPPC, DPPGNa, Cholesterol, KC1003 and DSPE-PEG (2000) in 54.3:8:30:7.5:0.3 molar ratio). The Hb mixture from above is added to the lipids and mixed on an orbital shaker for 2 hours.

The crude vesicles were then extruded under nitrogen at ambient temperature using first stacked 0.8/0.6 micron membranes (2 passes) and then 2 passes through 0.4/0.2/0.2 stacked membranes.

The extruded mixture was purified by use of Sepharose 4B-CL using PBS to elute the particles.

For measurement of oxygen loading and offloading (p50 values), samples were gas exchanged under visible light using pure oxygen.

TABLE 4
NaCl, Glucose and ATP Components of
Selected Specimens from Example 7

| | HPCD | mM | NaCl | Glucose | ATP |
|---|---|---|---|---|---|
| Sample ID | mg/mL | Phosphate | mg/mL | mg/mL | mM |
| KC240708-2 | 25 | 10 | 12.4 | 5.6 | 10 |
| KC240708-3 | 25 | 10 | 6 | 5 | 10 |
| KC240708-4 | 25 | 10 | 6.6 | 0 | 10 |
| KC240708-9 | 25 | 10 | 12.8 | 5.2 | 5 |
| KC240708-10 | 25 | 10 | 6 | 5 | 5 |
| KC240708-11 | 25 | 10 | 7 | 0 | 5 |
| KC240708-16 | 25 | 10 | 0 | 0 | 5 |
| KC240708-17 | 25 | 10 | 0 | 5 | 5 |

TABLE 5
Characteristics of EM with ATP as allosteric effector and metabolism additive

| | | | | | | % | | |
|---|---|---|---|---|---|---|---|---|
| | | | | | Zeta | MetHb in | p50 6.8 | p50 7.6 |
| Sample ID | [Hb] | EE % | Size | PDI | potential | unlocked | offloading | loading |
| KC240708-2 | 136.36 | 96.90% | 227 | 0.145 | −31.94 | 4.5% | 36.63 | 31.69 |
| KC240708-3 | 144.89 | 100.00% | 211.4 | 0.108 | −25.73 | 1.6% | 43.73 | 41.07 |
| KC240708-4 | 154.69 | 99.20% | 216.7 | 0.098 | −26.39 | 2.0% | 48.77 | 42.61 |
| KC240708-9 | 152.98 | 96.40% | 246.9 | 0.21 | −24.1 | 7.0% | 31.94 | 24.54 |
| KC240708-10 | 167.05 | 99.00% | 206.2 | 0.161 | −25 | 2.2% | 40.5 | 36.78 |
| KC240708-11 | 198.58 | 99.40% | 220.6 | 0.163 | −21.19 | 0.0% | 44.95 | 37.73 |
| KC240708-16 | 239.49 | 99.60% | 203.5 | 0.067 | −22.65 | 0.0% | 43.92 | 38.15 |
| KC240708-17 | 253.55 | 100.00% | 208.4 | 0.154 | −25.97 | 0.0% | 31.25 | 26.32 |

TABLE 6
ATP with MgCl2 as allosteric effector and metabolism additive

| | HPCD | mM | NaCl | Glucose | ATP | | other |
|---|---|---|---|---|---|---|---|
| Sample ID | mg/mL | Phosphate | mg/mL | mg/mL | mM | Other | Mg/mL |
| KC240708-14 | 25 | 10 | 6 | 5 | 5 | MgCl₂ | 0.7 |
| KC240708-18 | 25 | 10 | 0 | 5.6 | 10 | MgCl₂ | 5 |
| KC240805 -2 | 25 | 10 | 0 | 5 | 10 | MgCl₂ | 1 |

TABLE 7
Characteristics of EM with ATP and MgCl2

| | | | | | | % | | |
|---|---|---|---|---|---|---|---|---|
| | | | | | Zeta | MetHb in | p50 6.8 | p50 7.6 |
| Sample ID | [Hb] | EE % | Size | PDI | potential | unlocked | offloading | loading |
| KC240708-14 | 140.63 | 99.70% | 211.7 | 0.162 | −28.36 | 4.3% | 30.9 | 27.3 |
| KC240708-18 | 231.82 | 98.90% | 203.9 | 0.055 | −21.08 | 0.0% | 50.11 | 42.07 |
| KC240805-2 | 199.9 | 100.0% | NA | NA | NA | 6.1% | 55.48 | 46.96 |

### Example 8: NADP(H) as Allosteric Effector and Metabolism Additive

5 mL of purified hemoglobin at 5 mM in 10 mM phosphate buffer at pH 6.75 is filtered through a 0.1 micron filter. To the filtered Hb is added as appropriate (see Table) HPCD, Glucose, NaCl, NADP (H) and MgCl2. The mixture is sonicated for 1 minute in a bath sonicator and then filtered through a 0.05 micron syringe filter.

Into a 20 mL reaction vessel is weighed 240 mg of solid lipid mixture (DPPC, DPPGNa, Cholesterol, KC1003 and DSPE-PEG (2000) in 54.3:8:30:7.5:0.3 molar ratio). The Hb mixture from above is added to the lipids and mixed on an orbital shaker for 2 hours.

The crude vesicles were then extruded under nitrogen at ambient temperature using first stacked 0.8/0.6 micron membranes (2 passes) and then 2 passes through 0.4/0.2/0.2 stacked membranes.

The extruded mixture was purified by use of Sepharose 4B-CL using PBS to elute the particles.

TABLE 8
EM with NADP(H)

| | Phosphate | HPCD | NaCl | Glucose | mM | | |
|---|---|---|---|---|---|---|---|
| SAMPLE ID# | mM | mg/mL | mg/mL | mg/mL | NADPH | Other | mg/mL |
| KC240711-1 | 10 | 25.6 | 0 | 0 | 4.7 | NA | NA |
| KC240711-2 | 10 | 25 | 6.8 | 0 | 4.8 | NA | NA |
| KC240711-3 | 10 | 25 | 6 | 5 | 4.7 | NA | NA |
| KC240711-4 | 10 | 25 | 6 | 5 | 9.4 | NA | NA |
| KC240711-5 | 10 | 25 | 6 | 5 | 3.8 | MgCl₂ | 1.46 |

TABLE 9
Selected characteristics of NADP(H) containing EM

| | | | | | | % | p50 | p50 |
|---|---|---|---|---|---|---|---|---|
| | | | | | Zeta | MetHb in | pH = 6.8 | pH = 7.6 |
| SAMPLE ID# | [Hb] | EE % | Size | PDI | potential | Unlocked | unloading | loading |
| KC240711-1 | 189.2 | 100.0% | 215.8 | 0.09 | −17.31 | 0.0% | 32.97 | 28.25 |
| KC240711-2 | 191.34 | 100.0% | 205.8 | 0.151 | −29.06 | 3.1% | 31.58 | 27.92 |
| KC240711-3 | 165.32 | 97.2% | 211.4 | 0.105 | −20.91 | 3.3% | 34.09 | 28.93 |
| KC240711-4 | 162.78 | 96.6% | 209.2 | 0.067 | −29.76 | 4.8% | 33.75 | 29.5 |
| KC240711-5 | 187.93 | 96.7% | 207.2 | 0.088 | −24.25 | 2.4% | 31.38 | 27.52 |

TABLE 10
Additional Metabolic Compounds as Effectors-Composition

| | HPCD | mM | NaCl | Glucose | | other | MgCl2 |
|---|---|---|---|---|---|---|---|
| Sample ID | mg/mL | Phosphate | mg/mL | mg/mL | Other | Mg/mL | mg/mL |
| KC241014-1 | 25 | 10 | 5 | 5 | GTP | 2.5 | 0 |
| KC241014-2 | 25 | 10 | 5 | 5 | GTP | 5 | 0 |
| KC241014-3 | 25 | 10 | 5 | 5 | GTP | 2.5 | 0.4 |
| KC241014-4 | 25 | 10 | 5 | 5 | UTP | 2.5 | 0 |
| KC241014-5 | 25 | 10 | 5 | 5 | UTP | 5 | 0 |
| KC241014-6 | 25 | 10 | 5 | 5 | UTP | 2.5 | 0.4 |
| KC241014-7 | 25 | 10 | 5 | 5 | CTP | 2.5 | 0 |
| KC241014-8 | 25 | 10 | 5 | 5 | CTP | 5 | 0 |
| KC241014-9 | 25 | 10 | 5 | 5 | CTP | 2.5 | 0.4 |

GTP = Guanosine Triphosphate
UTP = Uridine Triphosphate
CTP = Cytidine Triphosphate

TABLE 11
Additional Metabolic Compounds as Allosteric Effectors

| | | | | | | % | | |
|---|---|---|---|---|---|---|---|---|
| | | | | | Zeta | MetHb in | p50 6.8 | p50 7.6 |
| Sample ID | [Hb] | EE % | Size | PDI | potential | unlocked | offloading | loading |
| KC241014-1 | 105.11 | 98.4% | 235.9 | 0.12 | −41.35 | 0.0% | 38.77 | 37.72 |
| KC241014-2 | 161.93 | 98.2% | 240.5 | 0.05 | −26.98 | 0.0% | 43.95 | 43.36 |
| KC241014-3 | 125.00 | 97.7% | 246.9 | 0.14 | −39.81 | 0.1% | 33.98 | 32.76 |
| KC241014-4 | 133.52 | 97.9% | 239.3 | 0.12 | −40.03 | 0.1% | 38.28 | 37.37 |
| KC241014-5 | 150.57 | 97.7% | 245.6 | 0.11 | −19.02 | 0.0% | 44.97 | 43.23 |
| KC241014-6 | 136.36 | 97.5% | 249.6 | 0.1 | −40.14 | 0.3% | 34.07 | 30.86 |
| KC241014-7 | 82.39 | 95.2% | 248.6 | 0.13 | −20.18 | 4.6% | 35.53 | 34.75 |
| KC241014-8 | 184.66 | 97.8% | 237.6 | 0.12 | −37.51 | 0.8% | 43.13 | 40.65 |
| KC241014-9 | 79.55 | 94.3% | 225.4 | 0.07 | −41 | 0.4% | 34.80 | 31.20 |

TABLE 12
Composition of Comparative Example with 2,3-DPG

| | HPCD | mM | NaCl | Glucose | 2,3-DPG |
|---|---|---|---|---|---|
| Sample ID | mg/mL | Phosphate | mg/mL | mg/mL | mg/ml |
| KC240708-22 | 25 | 10 | 6 | 5 | 2.2 |
| KC240708-21 | 25 | 10 | 13.2 | 5.4 | 2.4 |

TABLE 13
Comparative example with 2,3-DPG

| | | | | | | % | | |
|---|---|---|---|---|---|---|---|---|
| | | | | | Zeta | MetHb in | p50 6.8 | p50 7.6 |
| Sample ID | [Hb] | EE % | Size | PDI | potential | unlocked | offloading | loading |
| KC240708-22 | 158.52 | 99.50% | 225.2 | 0.114 | −31.61 | 0.0% | 35.87 | 31.96 |
| KC240708-21 | 181.11 | 97.60% | 220.3 | 0.131 | −28.23 | 2.4% | 33.86 | 27.07 |

Notwithstanding the specific embodiments, features, elements, combinations and sub-combinations disclosed herein, it is expressly considered and here disclosed that every single element, every single feature, and every combination and sub-combination thereof disclosed herein may be combined with every other element, feature, combination and sub-combination disclosed herein.

It will be appreciated by those skilled in the art that changes could be made to the embodiments described above without departing from the inventive concept thereof. It is understood, therefore, that this invention is not limited to the particular embodiments disclosed, but it is intended to cover modifications within the spirit and scope of the present invention as outlined in the present disclosure and defined according to the broadest reasonable reading of the claims that follow, read in light of the present specification.

1. A composition for the delivery of oxygen and metabolic factors/co-factors to organs in need due to insufficient ability of RBCs to perform these functions, the composition comprising a nanoparticle encapsulating an oxygen carrier and a metabolic agent.

2. The composition of claim 1, further comprising a lipid.

3. The composition of claim 1, wherein Hemoglobin is the oxygen carrier.

4. The composition of claim 1, wherein the metabolic factor is a nucleotide triphosphate.

5. The composition of claim 4, wherein the nucleotide triphosphate serves as an allosteric effector of hemoglobin in oxygen delivery.

6. The composition of claim 5, wherein the nucleotide triphosphate facilitates the release of oxygen in organs.

7. The composition of claim 5, wherein the nucleoside triphosphate is Adenosine triphosphate.

8. The composition of claim 5, wherein the nucleoside triphosphate is Guanosine triphosphate.

9. The composition of claim 1, wherein the metabolic factor is a nucleotide diphosphate.

10. The composition of claim 9, wherein the nucleotide diphosphate is Adenosine diphosphate.

11. The composition of claim 9, wherein the nucleotide diphosphate is Guanosine diphosphate.

12. The composition of claim 9, wherein the nucleotide diphosphate is NAD (H).

13. The composition of claim 9, wherein the nucleotide diphosphate is NADP (H).

14. The composition of claim 9, wherein the nucleotide diphosphate also serves as an allosteric effector of hemoglobin in oxygen delivery.

15. The composition of claim 1 in the form of a solid.

16. The composition of claim 1 in the form of an injectable suspension reconstituted from a lyophilized solid.

17. The composition of claim 1 further comprises a lyo-protectant.

18. The composition of claim 16 further comprising an encapsulated lyo-protectant.

19. The composition of claim 17 which is a saccharide, disaccharide, or polysaccharide.

20. The composition of claim 18 which is a saccharide, disaccharide, polysaccharide.

21-99. (canceled)
